(19) United States
(12) Patent Application Publication
(10) Pub. No.: US 2008/0200862 A1
(43) Pub. Date: Aug. 21, 2008

(54) Method To Administer Stem Cells In Combination With One Or More Acoustically Active Materials And Ultrasound Energy

(75) Inventors: Evan C. Unger, Tucson, AZ (US); Rachel LaBell, Vail, AZ (US); Terry O. Matsunaga, Tucson, AZ (US); Reena Zutshi, Tucson, AZ (US)

Correspondence Address: DALE F. REGELMAN, QUARLES & BRADY, LLP, ONE SOUTH CHURCH AVENUE AVE, STE. 1700, TUCSON, AZ, 85701-1621, US

(73) Assignee: IMARX THERAPEUTICS, INC., Tucson, AZ (US)

(21) Appl. No.: 11/718,882

(22) PCT Filed: Nov. 8, 2005
(86) PCT No.: PCT/US05/40659

§ 371 Date: May 8, 2007

Related U.S. Application Data
(60) Provisional application No. 60/626,363, filed on Nov. 8, 2004.

(51) Int. Cl. A61N 7/00; A61M 37/00

(52) U.S. Cl. 604/22; 604/518

(57) ABSTRACT

The invention comprises a method to administer stem cells to a patient in need thereof. The method provides acoustically active material, stem cells, and an ultrasound energy emitting device. The method administers the acoustically active material to the patient, administers the stem cells to the patient, and administers ultrasound energy to the patient using the ultrasound emitting device.

### FIELD OF THE INVENTION

This invention relates to delivery of stem cells in combination with ultrasound energy and one or more acoustically active materials.

### BACKGROUND OF THE INVENTION

Stem cells are unspecialized cells that have two important characteristics that distinguish them from other cells in the body. First, stem cells replenish their numbers for long periods through cell division. Second, after receiving certain chemical signals, stem cells can differentiate, or transform into specialized cells with specific functions, such as a heart cell or nerve cell.

Stem cells can be classified by the extent to which they can differentiate into different cell types. Totipotent stem cells can differentiate into any cell type in the body plus the placenta. A fertilized egg is a type of totipotent stem cell. Cells produced in the first few divisions of the fertilized egg are also totipotent.

Pluripotent stem cells are descendants of the totipotent stem cells of the embryo. These cells, which develop about four days after fertilization, can differentiate into any cell type, except for totipotent stem cells and the cells of the placenta. Multipotent stem cells are descendents of pluripotent stem cells and antecedents of specialized cells in particular tissues. For example, hematopoietic stem cells, which are found primarily in the bone marrow, give rise to all of the cells found in the blood, including red blood cells, white blood cells, and platelets. Another example is neural stem cells, which can differentiate into nerve cells and neural support cells called glia.

Progenitor cells (or unipotent stem cells) can produce only one cell type. For example, erythroid progenitor cells differentiate into only red blood cells. At the end of the long chain of cell divisions are “terminally differentiated” cells, such as a liver cell or lung cell, which are permanently committed to specific functions. These cells stay committed to their functions for the life of the organism or until a tumor develops. In the case of a tumor, the cells dedifferentiate, or return to a less mature state.

Perhaps the best-known stem cell therapy to date is the bone marrow transplant, which is used to treat leukemia and other types of cancer, as well as various blood disorders.

In a bone marrow transplant, the patient's bone marrow stem cells are replaced with those from a healthy, matching donor. To do this, all of the patient's existing bone marrow and abnormal leukocytes are first killed using a combination of chemotherapy and radiation. Next, a sample of donor bone marrow containing healthy stem cells is introduced into the patient's bloodstream.

While most blood stem cells reside in the bone marrow, a small number are present in the bloodstream. These multipotent peripheral blood stem cells can be used just like bone marrow stem cells to treat leukemia, other cancers and various blood disorders. Since they can be obtained from drawn blood, peripheral blood stem cells are easier to collect than bone marrow stem cells, which must be extracted from within bones. This provides a less invasive treatment option than bone marrow stem cells.

Newborn infants no longer need their umbilical cords, so they have traditionally been discarded as a by-product of the birth process. In recent years, however, the multipotent-stem-cell-rich blood found in the umbilical cord has proven useful in treating the same types of health problems as those treated using bone marrow stem cells and peripheral blood stem cells.

The United States National Institutes of Health has established a National Stem Cell Bank at the WiCell Research Institute in Wisconsin. The National Stem Cell Bank will consolidate many of the federally funded eligible human embryonic stem (ES) cell lines in one location, reduce the costs that researchers have to pay for the cells, and maintain quality control over the cells. The Stem Cell Bank will provide scientists affordable and timely access to federally approved human embryonic stem cells and other technical support that will make it easier for scientists to obtain the cell lines currently listed on the NIH Human Embryonic Stem Cell Registry.

What is needed is a method for targeted delivery of stem cells to a particular body site. Applicants' method comprises administering interarterially and/or intravenously stems cells in combination with one or more acoustically active materials. An ultrasound energy emitting device is disposed over a target body site. Acoustic sound waves are administered to that target body site during all or a portion of the time the stem cells and acoustically active materials are being administered.

### SUMMARY OF THE INVENTION

The invention comprises a method to administer stem cells to a patient in need thereof. The method provides acoustically active material, stem cells, and an ultrasound energy emitting device. The method administers the acoustically active material to the patient, administers the stem cells to the patient, and administers ultrasound energy to the patient using the ultrasound emitting device.

### DETAILED DESCRIPTION OF THE PREFERRED EMBODIMENTS

It is known in the art the use ultrasound energy to activate acoustic materials for cavitation, or to move the acoustic materials as with radiation force. Using Applicants' method, acoustically active materials are preferably associated with stem cells such that the stem cells become acoustically active, and can thereby be perturbed with ultrasound. The invention is useful for targeted delivery of stem cells to a particular body site.

The present invention is directed to targeted delivery of stem cells to treat disease. Current methods of delivery are either invasive or ineffectual. Delivery of the stem cells using acoustically active materials and ultrasound is less invasive and, because the delivery is targeted, more effective.

Various types of stem cells may be administered to a patient to treat a wide variety of diseases. The present invention is directed to delivering any of the various types of stem cells. Such cells include, but are not limited to, adult multipotent stem cells such as hematopoietic, mesenchymal, neural stem cells (both neuronal and non-neuronal cells), epithelial, and epidermal stem cells. Such cells also include, but are not limited to, embryonic stem cells. Embryonic stem cells are in one of the following forms: totipotent, pluripotent, or multipotent. The multipotent cells differentiate into specific cells. This invention encompasses delivery of undifferentiated or differentiated cells, including precursor and progenitor cells.

Diseases that can be treated using Applicants' method include, but are not limited to, autoimmune diseases (Lupus, Type I Diabetes, Multiple Sclerosis, Rheumatoid arthritis, HIV), cancer (ovarian, brain, breast, myeloma, leukemia, lymphoma), CNS (Parkinson's disease, Alzheimer's disease, Lou Gehrig's disease), and heart disease. Additionally, Applicants' invention encompasses treatment of injuries (such as spinal cord injuries) and birth defects.

Specifically, the present invention provides methods for stem cell delivery to a subject comprising directing acoustic energy, i.e. sound waves, to a target region of the patient's body, and concurrently administering to the patient acoustically active material (“AAM”) and the stem cells. In certain embodiments, the acoustic sound waves are focused. In other embodiments, the acoustic sound waves are non-focused.

In certain embodiments, insonation comprises using a continuous wave. In other embodiments, insonation is performed using one or more pulsed emission(s) of acoustic energy waves. The ultrasound applied in accordance with the inventive methods can range in frequency, intensity and mechanical index. In certain embodiments, the ultrasound energy ranges in frequency from about 100 kHz to about 20 MHz. In certain embodiments, ultrasound ranges in intensity from about 0.1 Watts/cm² to about 30 Watts/cm². In certain embodiments, a mechanical index ranges from about 0.1 to 2.

Applicants' method comprises a variety of embodiments for administering the acoustically active material, and/or the stem cells. In certain embodiments, the AAM is administered intravenously. In certain embodiments, the AAM is administered intra-arterially. In certain embodiments, the stem cells are co-administered with the AAM. In other embodiments, the stem cells are administered just prior to, simultaneously with, or following, administration of the AAM. In still other embodiments, stem cells are incorporated into the AAM for delivery. One of the advantages of the present invention comprises the capture of ultrasonic energy by the acoustically active material, causing cavitation and the rupture of the acoustically active material, thereby enhancing cellular uptake of the stem cells.

Acoustically active material can include, but is not limited to, microbubbles, nanobubbles, and nanodroplets. In certain embodiments, the AAM comprises a cationic material. In other embodiments, the AAM comprises an anionic material.

Microbubbles, nanobubbles and nanodroplets of the present invention can further include a targeting ligand that can promote targeting and selective binding to particular tissues in the body. A targeting ligand of the invention can specifically bind with brain endothelial cells, for example, by specifically targeting cell adhesion polypeptides (e.g., Integrin receptors). A targeting ligand can include, for example, a polypeptide selected from SEQ ID NO:1 (VLREGPAGG), SEQ ID NO:2 (CNSRLHRC), SEQ ID NO:3 (CENWWGDVC), SEQ ID NO:4 (CLSSRLDAC) or SEQ ID NO:5 (CRGDC).

A written Sequence Listing for the above-described targeting ligands is appended hereto. In addition, Applicants are providing that same Sequence Listing in Computer Readable Form encoded on a disk filed on even date with this Application. The information recorded in computer readable form is identical to the written Sequence Listing herein.

The following examples are presented to further illustrate to persons skilled in the art how to make and use the invention. These examples are not intended as a limitation, however, upon the scope of the invention, which is defined by the claims included herein.

### EXAMPLE 1

### Production Of Cationic Nanobubbles

A 10 mL volume of propylene glycol (Fisher Scientific) is disposed into a beaker and heated to 75° C. Distearoyl trimethylammonium propane (DSTAP, 6 mg, Avanti Polar Lipids, Alabaster, Ala.) is added and stirred until dissolved. Dipalmitoyl phosphatidylethanolamine-methoxy(polyethylene glycol)5000 (DPPE-PEG 5000, 40 mg, Avanti Polar Lipids, Alabaster, Ala.) is added and stirred until dissolved. Finally, dipalmitoyl phosphatidylcholine (DPPC, 54 mg, Avanti Polar Lipids, Alabaster, Ala.) is added and stirred until dissolved. In a separate beaker, glycerol (5 mL, Fisher Scientific, Hampton, N.H.) is combined with 18 MOhm water (85 mL, Bamstead International, Dubuque, Iowa) and heated to 55° C. Sodium chloride (0.48 g, Aldrich, Milwaukee, Wis.) is added into the water/glycerol solution and stirred until dissolved. Sodium monobasic phosphate (0.23 g, Spectrum, New Brunswick, N.J.) is added and stirred until dissolved. Lastly, sodium dibasic phosphate (0.22 g, Spectrum, New Brunswick, N.J.) is added and stirred until dissolved. Finally, the propylene glycol suspension is poured into the water solution with vigorous stirring until the suspension was homogenous. The compounded lipid suspension is stored at 4° C. until used for nanoparticle formation.

A plurality of vials are filled with 1.6 mL of lipid suspension. The headspace is filled with perfluoropropane by cycling vacuum and gas fill five times. Thereafter, the vials are stoppered, crimped closed, and stored in a refrigerator. The headspace is sampled and analyzed for perfluoropropane content.

Prior to use a vial is removed form the refrigerator and allowed to warm to room temperature. The vial is activated in a shaker for 45 seconds with a speed of 4500 rpm. The activated vial is allowed to sit on the bench for 15 minutes and then inverted gently 10 times to ensure a homogenous mixture.

### EXAMPLE 2

### Incubation Of Stem Cells With Materials From Example 1

Stem cells obtained from the National Stem Cell Bank are washed with phosphate buffered saline, and placed in cell culture media comprising the nanobubbles of Example 1. Microscopic examination shows a substantial number of nanobubbles are taken up by the cells.

### EXAMPLE 3

### Radiation Force Movement Of Stem Cells Containing Nanobubbles

Nanobubbles impregnated with cationic lipids (i.e. DOTAP or other cationic lipids, Vical, San Diego, Calif.), and DSPE-biotin, are incubated with stem cells for one hour followed by suspending the cells and centrifuging to remove excess cationic lipids. The cell/nanobubble mixture is then passed through a tube previously affixed with neuralite avidin. The stem cells/fluorescent cationic nanobubbles are then passed through the tube at 1 ml/min. The tube is exposed to ultrasound energy. A 10-MHz center frequency, high-power, single-element transducer was used. The driving wave was a 10 MHz, 40-cycle sinusoidal pulse with peak negative pressure of 1.59 MPa or 2.22 MPa.

Insonation occurred as the bubble/cell affixed mixture passed through the tube. Absorption was then monitored at 510 nm. It was found that the cells were affixed vs. control (no insonation) as determined by the increase in fluorescence over the tube.

### EXAMPLE 4

### Production Of MRX-815, Incubation With Polylysine And Stem Cells

A lipid mixture comprising 60 mg dipalmitoyl phosphatidic acid [DPPA], 540 mg dipalmitoyl phosphatidylcholine [DPPC], and 400 mg of dipalmitoyl phosphatidyl ethanolamine polyethyleneglycol-5000 [DPPE-PEG-5000] is formed by sequential dissolution in 100 mL propylene glycol at 60° C. This solution is brought up to one liter with 850 mL normal saline and 50 mL glycerol at room temperature.

Vials containing 1.5 mL of lipid solution with a headspace of perfluoropropane are shaken at 4200 rpm for 45 seconds to activate microbubbles. A mixture comprising 0-200 mg of polylysine in saline:propylene glycol:glycerol (85:10:5) is added to the vial, and the complex incubated for 30 minutes at room temperature. Activated microbubbles-polylysine complex is then added to hematopoietic stem cells (HSCs) in tissue culture media and allowed to incubate overnight at 37° C.

Infusion of such a delivery agent through a catheter, followed by ultrasound treatment over the target site to cavitate the bubbles enables the uptake of the genetic material at the target site.

### EXAMPLE 5

### Delivery Of Nanobubbles-Ferridex Complex Into Stem Cells

Ferridex, i.e. superparamagnetic iron oxide (SPIO) nanoparticles, at a concentration of 100 μg/mL is added to a flask containing 3-10 μg/mL of protamine sulfate and shaken for 5-10 min to form the complex. The complex is added to a solution of nanobubbles and incubated with shaking for 30 minutes. The nanobubbles-Ferridex complex is added to cell culture media containing stem cells and incubated overnight at 37° C. for a ferridex-complex final concentration of 50 μg/mL.

### EXAMPLE 6

### Delivering Stem Cells Using Liquid Perfluorocarbon Containing Cationic Nanoparticles

A microbubble formulation was prepared using two steps, namely the compounding of the lipids into suspension, followed by the formation of the nanoparticles with perfluorohexane. The microbubble formulation of this Example 6 has a lipid ratio of 2:1 1,2-dioleoyl-trimethylammonium-propane (DOTAP): L-α-dioleoyl phosphatidylethanolamine (DOPE) with an additional 5% 1,2-dioleoyl-SN-glycero-3-phosphoethanolamine-N-[methoxy(polyethylene glycol)-2000] (mPEG2000 PE).

A beaker of saline (300 mL) was heated to 50° C. The DOPE (100 mg, Avanti Polar Lipids, Alabaster, Ala.) was added followed by DOTAP (200 mg, Avanti Polar Lipids, Alabaster, Ala.) and lastly mPEG2000 PE (15 mg, Avanti Polar Lipids, Alabaster, Ala.), and the suspension was stirred for 2 hours. The suspension was homogenized on a Silverson L4RT with a 1inch tubular mixing unit with a square-hole high shear screen (Silverson Machines LTD, East Longfellow, Mich.) homogenizer at 7500 rpm for 10 minutes. After homogenization the suspension was translucent and homogenous. The lipid suspension was QS to 300 mL and stored in the refrigerator before next step.

The cold suspension was put in an ice bath and homogenized on a Silverson at 7500 rpm during a dropwise addition of cold perfluorohexane (6 mL, Aldrich, Milwaukee, Wis.). The suspension was homogenized for 30 min. after addition of perfluorocarbon. Lastly, the suspension was extruded through 47 mm polycarbonate membranes (Whatman, Clifton, N.J.) with 100 nm pore size using an Emulsiflex C5 (Avestin, Ottawa, Ontario). The resulting formulation (1.5 mL) was pipetted into 2 ml glass vials, stoppered, and crimped closed. The formulation was stored at 4° C.

Human mesenchymal stem cells (MSCs; Cambrex, Baltimore, Md.) were grown to 80% confluence in the recommended media. The microbubble formulation was added directly to the media containing stem cells, and the cell suspension incubated overnight at 37° C. The stem cells were then delivered either through a catheter directly to disease site, or administered intravenously and targeted for localized delivery using ultrasound energy emission(s).

### EXAMPLE 7

### Nerve Stem Cells Incubated With Nanobubbles

Neural stem cells from fetal nerve cells of a human brain were obtained and complexed to nanobubbles as described hereinabove. The bubbles/stem cell complex is then infused via catheter directly into the internal carotid artery of a 60 year old male afflicted with Parkinson's disease as diagnosed by pill-rolling (bradykinesia) behavior. Immediately downstream in the region of the carotid artery and near the temporal lobe is placed a non-focused ultrasound transducer. A 1 MHz insonation pulse is then applied for the duration of the infusion. This results in an opening of the gap junctions between the endothelial cells followed by radiation force-induced diffusion of the stem cells.

The cells, once introduced into the third ventricle space, migrated to the substantia nigra and transformed into mature brain cells that produced/released Dopamine. After six months, the patient's bradykinesia began to resolve followed by a normal gait upon ambulation.

### EXAMPLE 8

### Regeneration Of Cardiac Myocytes Via Stem Cell—Nanobubble Complexation And Ultrasound Therapy

A 70 year old male with multiple myocardial infarcts in both the antero-lateral and postero-lateral walls of the myocardium is admitted for evaluation. Upon ultrasound, the patient is found to have only a 20% ejection fraction. The patient experiences significant shortness of breath at rest and is on maintained on continual 100% O₂ via nasal cannula to maintain reasonable saturation.

The patient presents to the cardiac catheterization laboratory where he is catheterized through the coronary sinus. The patient is then infused with nanobubble —embryonic stem cell complexed mixture through the sinus followed by application of 1 MHz ultrasound at 1 W/cm² and a 20% duty cycle through the intercostals space and directly on the LAD and left circumflex branch. After 30 minutes of insonation, the ultrasound is removed and the catheter withdrawn. The patient remains stable.

Six months later, the patient returns to the hospital and an echocardiogram is conducted. The patient is found to have an ejection fraction of 55% and no longer requires continual O₂ by nasal cannula. The patient also has increased his exercise to full ambulation without SOB.

While the preferred embodiments of the present invention have been illustrated in detail, it should be apparent that modifications and adaptations to those embodiments may occur to one skilled in the art without departing from the scope of the present invention as set forth in the following claims.

We claim:

1. A method to administer stem cells to a patient in need thereof, comprising the steps of:
providing acoustically active material, stem cells, and an ultrasound energy emitting device;
administering said acoustically active material to said patient;
administering said stem cells to said patient; and
administering ultrasound energy to the patient using said ultrasound emitting device.

2. The method of claim 1, wherein said administering ultrasound energy step further comprises administering ultrasound energy comprising a frequency from about 100 kHz to about 20 MHz.

3. The method of claim 2, wherein said administering ultrasound energy step further comprises administering ultrasound energy comprising an energy level from about 0.1 Watts/cm² to about 30 Watts/cm².

4. The method of claim 3, wherein said administering ultrasound energy step further comprises administering ultrasound energy comprising a mechanical index from about 0.1 to about 2.

5. The method of claim 1, wherein said providing acoustically active material step further comprising the step of providing acoustically active material comprising a targeting ligand.

6. The method of claim 5, wherein said providing acoustically active material comprising a targeting ligand step further comprises providing acoustically active material comprising a targeting ligand that specifically binds with brain endothelial cells.

7. The method of claim 5, wherein said providing acoustically active material comprising a targeting ligand step further comprises providing acoustically active material comprising a targeting ligand comprising a polypeptide.

8. The method of claim 7, wherein said providing acoustically active material comprising a targeting ligand comprising a polypeptide step further comprises providing acoustically active material comprising a targeting ligand comprising SEQ. ID. 1.

9. The method of claim 7, wherein said providing acoustically active material comprising a targeting ligand comprising a polypeptide step further comprises providing acoustically active material comprising a targeting ligand comprising SEQ. ID. 2.

10. The method of claim 7, wherein said providing acoustically active material comprising a targeting ligand comprising a polypeptide step further comprises providing acoustically active material comprising a targeting ligand comprising SEQ. ID. 3.

11. The method of claim 7, wherein said providing acoustically active material comprising a targeting ligand comprising a polypeptide step further comprises providing acoustically active material comprising a targeting ligand comprising SEQ. ID. 4.

12. The method of claim 7, wherein said providing acoustically active material comprising a targeting ligand comprising a polypeptide step further comprises providing acoustically active material comprising a targeting ligand comprising SEQ. ID. 5.

13. The method of claim 1, wherein said stem cells and said acoustically active material are administered concurrently to said patient.

14. The method of claim 1, wherein said stem cells, said acoustically active material, and said ultrasound energy, are administered concurrently to said patient.

15. The method of claim 1, wherein said providing acoustically active material further comprises the steps of:
disposing L-α-dioleoyl phosphatidylethanolamine, 1,2-dioleoyl-trimethylammonium-propane, and 1,2-dioleoyl-SN-glycero-3-phosphoethanolamine-N-[methoxy(polyethylene glycol)-2000, in saline; and
adding perfluorohexane to the saline suspension with mixing.

16. The method of claim 1, wherein said providing acoustically active material further comprises the steps of:
adding distearoyl trimethylammonium propane to propylene glycol in a first vessel with stirring until dissolved;
adding dipalmitoyl phosphatidylethanolamine-methoxy(polyethylene glycol)5000 (DPPE-PEG 5000 to said first vessel with stirring until dissolved;
adding dipalmitoyl phosphatidylcholine to said first vessel with stirring until dissolved;
mixing in a second vessel glycerol and 18 MOhm water;
adding sodium chloride to said second vessel with stirring until dissolved;
adding sodium monobasic phosphate to said second vessel until dissolved;
adding sodium dibasic phosphate to said second vessel until dissolved;
adding the propylene glycol solution from said first vessel to the aqueous solution in said second vessel with vigorous stirring until the resulting lipid suspension is homogenous;
disposing said lipid suspension in a vial;
filing the head space of said vial with perfluoropropane;
stoppering said vial;
shaking said vial for 45 seconds at a speed of 4500 rpm.

17. The method of claim 16, wherein said providing stem cells step further comprises:
forming a microbubble-polylysine complex by adding polylysine in saline:propylene glycol:glycerol (85:10:5) to said vial after said shaking step;
adding said microbubble-polylysine complex to hematopoietic stem cells (HSCs) in tissue culture media;
incubating said microbubble-polylysine/hematopoietic stem cell mixture overnight at 37° C.

18. The method of claim 17, further comprising the step of adding superparamagnetic iron oxide particles to said microbubble-polylysine/hematopoietic stem cell mixture before said incubating step.

19. The method of claim 1, wherein said providing stem cells step further comprises the step of growing Human mesenchymal stem cells to 80% confluence.

20. The method of claim 19, wherein said administering said stem cells to the patient further comprises administering the Human mesenchymal stem cells to said patient via a catheter.

21. A method to treat a patient suffering from Parkinson's disease, comprising the steps of:
providing nanobubbles comprising 1,2-dioleoyl-trimethylammonium-propane;
mixing neural stem cells from fetal nerve cells of a human brain with said nanobubbles;
infusing said nanobubbles/stem cell mixture via a catheter directly into the internal carotid artery of said patient;
disposing on said patient a non-focused ultrasound transducer immediately downstream in the region of the carotid artery and near the temporal lobe;
administering to said patient a 1 MHz insonation for the duration of the infusion.

22. A method to treat a patient having multiple myocardial infarcts, comprising the steps of:
providing a nanobubble-embryonic stem cell mixture;
providing an ultrasound energy emitting device;
catheterizing said patient through the coronary sinus;
infusing said patient with said nanobubble-embryonic stem cell mixture;
insonating said patient using said ultrasound emitting device through the intercostals space using an energy level of 1 W/cm² and a 20% duty cycle and a frequency of 1 MHz.


SEQUENCE LISTING (5 sequences)

SEQ ID NO: 1 - protein, 9 aa, Artificial Sequence
  Chemically Synthesized
VLREGPAGG

SEQ ID NO: 2 - protein, 8 aa, Artificial Sequence
  Chemically Synthesized
CNSRLHRC

SEQ ID NO: 3 - protein, 9 aa, Artificial Sequence
  Chemically Synthesized
CENWWGDVC

SEQ ID NO: 4 - protein, 9 aa, Artificial Sequence
  Chemically Synthesized
CLSSRLDAC

SEQ ID NO: 5 - protein, 5 aa, Artificial Sequence
  Chemically Synthesized
CRGDC